(19) United States
(12) Patent Application Publication
(10) Pub. No.: US 2004/0152178 A1
(43) Pub. Date: Aug. 5, 2004

(54) Adjuvant for accelating an enzyme activity

(76) Inventor: Yukio Matsumoto, Tokyo (JP)

Correspondence Address: Jay L. Chaskin, P.O. Box 1602, Fairfield, CT, 06825, US

(21) Appl. No.: 10/757,016

(22) Filed: Jan. 14, 2004

(30) Foreign Application Priority Data
Jan. 30, 2003 (JP) 2003-060659

(51) Int. Cl. C12N 9/00

(52) U.S. Cl. 435/183

(57) ABSTRACT

The present novel material, an adjuvant for accelating enzyme activity, is manufactured by contacting with organic substances and/or orgnic compounds included a small quantity of inorganic substances and catlizers including enzymes in nature, clay catalizer, microorganism group and water, and closely resembles to maternal amniotic liquid contained 15 kinds of specific metalic cation, which are indispensable material to activate the enzymes, and 20 kinds of low molecular organic compound contained indispensable basic amino acids, which is necessary to supply the biological components.

The novel material, accordingly, provides many functions which decompose and digest away the living or business refuses, comprising organic substances and/or organic compounds, and which protect and recover the bioorganisms and their damages, just same as the functions of the maternal amniotic liquid.

### CROSS-REFFERENCE TO RELATED APPLICATION

This intention claims priority under 35 U.S.C.S. 119 to Japanese patent application No.2003-060659 filed on Jan. 30, 2003, the entire contents of which is hereby incorporated by reference.

### BACKGROUND OF THE INVENTION

This invention relates to an adjuvant for accelating an enzyme activity and more particularly a novel adjuvant which can be induced from the organic substances or the organic compounds, under contacting with microorganism group in nature, clay catalyzers, enzymes and water, and which can revive a deteriorated living environment and biomechanism to their mormal condition, and improve excellently the functions of the living or biorogicl body tissue.

Customary, it has been already disclosed in Japanese patent application publication No. Kokai Hei 4-238887 and Hei 4-265286 that an activated humic agricaltural material, a seed bacillus liquid, agricaltural fertilizer and soil activater have been manufactured from organic substances or organic compounds under contacting with the catalizers including microorganisms in nature, clay catalizers, etc.

However, nowaday, the globe and the organic system or organism living on it, has fallen ill respectively, and they have come to a worst situation that it could not been restored by their management and restration fculties only furnished naturally in their evolution.

One of those origins results in the living refuse and sewage, which is discharged plentifully from the human society into the natural world, in accordance with development of the world civiliztion. The living refuse is presently burned or molten to treat, however, the treatment discharges heat and bad material into the air, and the bad material such as it's residue or ash, etc., have to be buried under the ground to treat them.

The dirt or filthy water discharged from men and beasts, can not be treated completely by any latest techniques, and consequently they are buried under the ground, or discharged into the river, as the burned ash or diluted non-toxic solution.

Those discharged materials are still in existence to accumulate in a form of a harmful consentrated substance, which can not be treated away by a force of nature, after dissolved into a sea, a river or a lake through the global cycle system, and they are taken into the human body through a drinking water or a food, and some of them, which can not be decomposed by human functional mechanism, are accumulated and condensed therein.

Once, the human living refuse, the dirt or filthy water dischrged from men and beasts on the globe, have been completely consumed by contacting with water, mineral soils, microorganisms, etc., created in the global system, by mutual supply and consumption under their symbolism, and a balanced harmless environment between supply and comsamption in nature has been maintained consequently.

However, a great deal of the discharges from the men and beasts are now destroying not only the balance between the above natural supply and comsomption systems but also a human fundamental physiological function.

Many activated mterials and techniques, which can maintain such normal cycle systems, have been provided for the persons skiled in the art, in view of those considerations, however, those arts have not given any satisfctory results so far.

In such prior arts, for example, Patent application publication No. Hei 4-238887, discloses, for above object, an activative humus agricaltural material comprising an aged mixture of an agricaltural waste mterial, a livestock raising waste material, a microorganism activated catalizer (a mixed stractural rock mineral) and diatomaceous earth, and also Patent application publication No. Hei 4-265286 describes, for a same object, a soil microorganism group and their metabolic materials extracted from a highly aged compost, and an activated soil microorganism group and concentrated extracts from their metabolites, which include an effective component aquatic solution of the mixed stractural rock mineral for activation of the microorganism group, and discloses also that those substances are used as seed bacillis, crop fertilizers or soil activaters.

However, those activated hemic agricaltural materials could achieve an expected purpose but they did not prepare actually a natural enviromental purification cycle faculty such as functions for extinction of the living organic waste, the discharged sludges and the filthy waters, and for recovery of physiological fundamental function in the vital system.

Accordingly, it is an object of the present invention to provide with an adjuvant for accelating enzyme activity providing natural purifiction cycle ability, which could not been setteled so far by the prior art.

It is an other object of the present invention to provide with an adjuvant for accelating enzyme activity providing a faculty closely related to an ability for recovery of physiological fundamental function in the vital system.

It is further object of the present invention to provide with an adjuvant for accelating enzyme activity manufactured from the biomass.

It is futher more object of the present invention to provide with an adjuvnt for accelating enzyme activity manufactured from the bioresidue.

Means for Solving Problem

(A) Fundamental Human Physiological Function and Enzyme Action

Originally, it is evidently that existence of microoganism, clay catalizer, biocatalyst (enzyme) and water prepared in the natural world, is indispensble to prification of the global environment, as disclosed in the above two prior patent applications, while, cast and action of the enzyme in a phenomenon that a life body concerned, is also indispensable respectively, as same as above reaction.

same as above reaction.

In other words, the life is very closely connected with the enzyme and therefore a ploblem on it's origine relates to principally the same of enzyme it'self.

The enzyme can be produced by very complex system only, which is directly produced from a living body or living cell.

Accordingly, it is necessary to exist all enzymes of all systems for formation of an individual enzyme molecule.

Those enzymes are useful to supply energy, which is necessary on formation or synthesis for all kinds of components of a nucleic acid, relating to formation or synthesis system of an amino acid, which becomes a material of an enzyme protein.

All biocells are generally organized from many same materials, however, in many organisms, same conjugated cooperative factors, prosthetic molecular group, and essentially same enzymes, etc., are discovered also.

The organism it'self has to receive many important ingredients of many enzymes from the others, in many times, because the organism can not synthesize them directly.

(B) Mechanism of Enzyme Reaction

The enzyme, which cn not be self-synthesized in the organism, can induce, at high speed without any side reaction, a chemical reaction which is difficult to induce with the chemical rocess.

Such reaction takes place by catalytic action of the enzyme and exchangs electron between a ractive substrate (S) and an enzyme (E) to induce some polarization or stress in the former, and finally it is activated. An oxidoreductase, a transferase and a hydrolase, etc., are classified into such enzymes, which cause those actions.

In the other side, fifteen kinds of metalic cation having 1 to 3 atomic valency, such as Na, K, R_{b} . . . Nᵢ . . . Cᵣ . . . Aₗ . . . strongly affects to activation of the enzyme and to production of the present adjuvant for accelating enzyme activity.

The mechanism of specific enzyme activation by those metalci cation can be explained as follow;

(1) The metalic cation makes an orgnic molecule connect to a protein.

(2) The rection is affected strongly by PH.

(3) Inactivated metalic cation is substituted.

(4) The substituted and inctivated metalic cation is activated so that it can connect to an effective position.

While, the anions such as chlorine ion, bromine ion, crbonic acid ion, citric acid ion, take part in the activation of enzymes also, however, the activation of those anions generally are unspecific in many cases, and the enzyme, in this case, shows activity in a certain extent, without any electrolyte.

It is clearly recognized that the enzyme is activated by the connection with such ions, especially under an alkalinity PH, also.

(C) Formaton of the Enzyme

The enzyme, which contacts with a reactive substrate, after activated, as mentioned above, must be synthesized continuously, and this indispensable material for an organism has to increase always, according to it's growth, in proportion with increased volums of the organism.

However, the enzymes have to be synthsized continuously because they are also decomposed in some rates, even if they don't grown up.

Accordingly, the life can not be maintained without the continuous synthesis of the enzyme, and the synthsis of the activative enzyme must be a reaction accompaning the synthesises of protein portion and a prosthetic group, in a certain case.

However, a certain prosthetic group must be supplied from outside because the organism can not self-synthsize all indispensable prosthetic groups, and further the enzyme has to be supplied also a special metal if the former includes the later.

The supply of those prosthetic groups and the special metals is one of the indispensable conditions for the present novel material formation.

While, the reaction for enzyme formation is a self-catalitic reaction, and onece the enzyme is activated by H ion, etc., it increases rapidly under the self-contact.

It should be also considered that the formation of the enzyme is controlled by a gene and a metabolic material.

(D) Activating Mechanism of the Enzyme by a Metalic Ion

A metal (M) forms an indispensable portion of an active center of the enzyme, and acts also as a connecter to hold the substrate in the active center of the enzyme, combinning with both of the enzyme and the substrate

The connecting sequences of those factor are as follow:

(1) E+M+S=E M S (Complex)

(2) E+M=EM (Activated enzyme)

EM+S=E M S (Complex)

(3) M+S=M S (Actual sabstrate)

E+M S=E M S (Complex)

However, an enzyme reaction is necessary occasionally the enzyme (E), the substrate (S) and other material (cooperative fucter) such as the metal cations and the inorganic anions described thereabove to progress the reaction.

### SUMMARY OF THE INVENTON

According to above studies and considerations firstly a bioprotoplasm such as biomass, organic substance in the organic residue, or organic compound including a little inorganic compound, which is shown as a general formula, CₐH_{b}O_{c}N_{d}PₑS_{f}, are used as a raw material in a process for the present adjuvant for accelating enzyme activity, and secndly added biocatalizers, for example, enzyme catalizers living in nature, cray ctalizers, microorganism group and water thereto.

The mixture thereafter is started to react under a specific condition such as an atomospheric pressure and an anaerobic condition, and oxidated imperfectly under aerobic condition, through periodrical aerobic condition, to yield a substance, which is quite alike to amniotic liquid of a mather's body in it's ingredients and effects, described thereinafter.

### DETAILED DESCRIPTION OF THE INVENTION

### EXAMPLE

4 tons per one day of biomass or biological residue presented as a general formula [OₐH_{b}O_{c}N_{d}PₑS_{f}][but _{a-f}; positive number] were charged into a reactor received enzyme catalists living in nature, clay ctalizer, microorganism group and water, and reacted for 10 days under an atomospheric pressure and 23.3° C.-24.3° C.

2.4 tons per a day of a novel djuvant for accelating enzyme activity, which is quite alike to biological amniotic liquid, were obtaied in the rate of 60% yield.

The novel material contained 15 kinds of inorganic materials (A) and low molecular organic material (B) including 20 kinds of indispensable basic amino acid, as shown in Tables 1 and 2, according to a quantum measurment.

However, R_{b}, Cₛ, and Nᵢ of the 15 kinds of inorganic materials in the Table don't be discribed for their contaents are trace.

The above reaction was also carried out in seven reactors through the following process as shown in Table 3 to 6.

First Process: Partial Decompositon of an Organic Component, [CₐH_{b}O_{c}N_{d}], Under an Anaerobic Conditon. (First Reactor α)

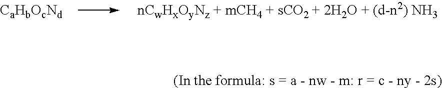

Conditions in the reaction: Normal Atomosphere

ORP: −409 −472

PH: 7.61˜7.78

Temperature: 23.2˜24.3° C.

Second Process: Forcible Intermitent Aerobic Process (Process that Nitrogen Does not be Oxidized Until Nitric Acid) (Second Reactor β)

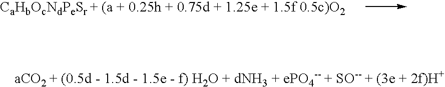

Condition in the Reaction:

ORP: 145˜20

DO: 0.39˜0.72

PM: 8.02˜8.30

Temperature: 26.5=18 27.9° C.

Third Process: Imperfect Oxidation Process of an Orgnic Compound Under Aerobic Condition (Seventh Reactor γ)

(P and S protion in natural organic compound are neglected to show in the table because they are very little (Trace) in comparison with the other atoms)

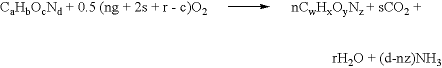

(In the formula:

r=0.5[b−mx−3(d−nz)]

s=a−nw)

Condition in the Reaction:

ORP: 53 84

DO: 0.30˜0.81

PH: 7.41˜7.65

Temperature: 20.0˜21.1° C.

Ingredients and Special Characters of the Present Adjuvant for Accelating Enzyme Activity

The ingredients and special characters of such novel material resemble very closely with them of maternal amniotic liquid, which is indispensable not only in the steps of biological growth and functional development of the fetus but also as an information source of it's mature degree and it's certain suffering.

The ingredients, as same as the amniotic liquid, in the other side, include proteins organizing an amino acid and electrolyte, and further include bioinorganic compound expect for an essential amino acide, of which an organism can not produce, and an electrolyte which is necessary for activating of the organism. (Reffer; Tables 1 and 2)

The new material showed a light yellow color and weak alkalinity in the range of PH 7.14 to 7.65, as same as the amniotic liquid, on and after the middle term of fatus growth.

Accordingly, it provided many functions for improvements of the biological growth, biological suffering and physiological operation, and also functions for decompositon and digestion of non-biological organic or inorganic compounds, just sme as the amniotic liquid, as described hereinafter,

Utilizations and Effects of the Novel Material

(1) Decompositon and Digestion of the Non-Biological Organic or Inorganic Compounds

(a) Treatment of the Home Raw Refuse With the Novel Material

| | Prior | |
|---|---|---|
| | material | Present new material |
| Initial charge of basic material Charge of home raw refuse Total charge Discharge Discharged basic material Term of treatment | 20 l 0.7 Kg/day 210 Kg 45 l 40 Kg 10 Months | 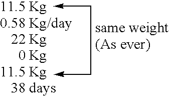 |

In the above experiment, 22 Kg of the home raw refuse has been completely decomposed by the present novel material to null through 38 days, although it's initial charge has not been changed at all, while the prior treatment could not digested completely even after 10 months, and remained therein 45 l of the residue.

(b) Treatment of the Business Raw Refuse With the Present Novel Material

| | |
|---|---|
| Initial charge of basic material Charge of business raw refuse Total charge Discharge Discharge of basic material Term of treatment | 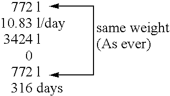 |

In this run, the discharge of the basic material has been no charge after the treatment but the chrged raw refus has been completely decomposed and digested after 316 days.

(c) Treatment of the Drainage With the Present Novel Material

A proper quantity of the present novel material were added to the drainage, which contains 12,000 mg/1 of mixed floating materials, and 95% or more of the later have been decomposed after 20 days.

In this reaction, a proper quantity of the drainages have reduced throughly in comparison with the basic wter.

Accordingly, a completely closed circulation system can be employed in this treating system because the drainage can be recycled as a diluent of the basic water, without discharging at all.

2) Improvement for Physiologicl Function of the Organism by the Present Novel Material

The present novel material provides many effects for improvement of hypofunction for endocrine and immunity systems and for prevention and improvement of healing ability of the life-style related diseases nd the chronic disease, because it has a same property as the mniotic liquid in a mathers body and therefore provides clearly a physical improving function also, as follow;

(a) For Diabetes and Hyperglycemia

### Medical Examinations: 30 Instances: The male Sex, 70%; The Female Sex, 30%

Those testers, who has had blood glucase rates in 200-500 mm/dl, was dosed 30 cc/day of the present novel material, and resulted in the rates of 120-130 mm/dl, after 30 days, respectovely.

(b) For Diabetcs and Eyesight Improvement (Dosage Instance for Hyperglycemi)

(1) The male sex, 57 years old, was very difficult to look his around, due to the heavy hyperglycemia but his condition of the disease recovered completely after dosages for 30 days.

(2) The male sex, 67 years old, was almost in loss eyesight and was in the depth of the despair but recovered such that he can look the thing clerly after drinking the present novel material for some months.

(c) For Cataract

The female sex, 70 years old, and other some instances, recovered by direct eyelotion of the present novel mterial through 10 days such that she can look clearly.

(d) For Agastric Cancer

Hairs of a male sex treating medically the gastric cancer, did not fll out for usage of the present novel material, even if he was dosed an anticancer drug material and a patient told that his life expectancy is a half year, could live long for 6 years with the present novel material, also.

(e) For Colonic Polyp

The male sex, 43 years old, discovered in the large intestine a polyp has been doesed the present novel material for six days and recognized the poryp has disappeared therefrom.

About ten other instances of the same symptom were recognized also under the same dosages.

(f) For Malignant Lymphoma

The female sex, 42 years old, recovered to a healthy condition under dosages of the present novel material for almost 3 months, without radio-therapy.

A blood test shown normal.

(g) For Leukemia

The female sex, 36 years old, was sentenced a half year of her life long expectance from a doctor, however, she could live long for six years by continious dosage in the amount of 30 cc per day of the present novel material.

(h) For Hepatitis C Virus (HCV)

The female sex, 47 years old, could not recover her condition of the disese in spite of a long meical tretment, however, a blood test after dosages of the present novel material for three months, shown remarkable improvement.

(i) For Cardiac Disorder

The male sex, 50 years old, was recognized a disorder on a left coronary aortic, but he recovered normally his health by dosges of the present novel material and became a cheerful man, which can work ordinarily.

(j) For Gliston

A male sex, 60 years old, had to undergo an operation for his galiston diseage, however, the operation has not been necessary due to dissolution of the galiston by dosages of the present novel material. Several same results appeared on the other paitient under the same condition.

(k) For Rheumatism

A male sex, 65 years old, recovered such that he can walk confortably after continuous dosage of 30 cc per day for one month, even if he was difficult to walk initially.

(l) For Skin Restoration

A cut end of the navel string of a neomate did not dry and was wet long time, but it has got well completely with a gauze atring of the present novel material.

(m) For Restoration of the Deep Scar

Each deep scar reached to a bone of two male sexes, 50 and 30 years old resectively, restored together with disappearance of it's pain by a gauze of the novel material for about 5 days.

(n) For Restoration of Bruise and Distortion

Those pains disappeared by a gauze stupe without appearance of cyanosis on the stupe portion.

There are twenty successes on such medical treatment so far.

(o) For Restoration of the Serious Athlete

The male sex, 50 years old, restored completely the serious athlete by bathing in 100 cc/200 l solution of the present novel mterial.

### EFFECTS OF THE INVENTION

The process of present novel material namely an adjuvant for accelating enzyme activity, providing above properties and functions, is very simple and low cost because it's production material is raw life refuse and raw business refuse, and it's apparatus is also multiple catalitic reactors only, which receive biocatalizers nd microorganisms in nature, clay catalizer and water, while, the novel adjuvant can decompose and digest the agricaltural and livestock rising waste materials, the life and business raw refuses, the filethy and muddy waters, without producton of the residues and drainages through the reactions, because the yield resembles very closely to amniotic liquid of the biological mather's body of which includes very much the indespencible components for biophysiological mechanism.

Accordingly, the treatment of those refuses or wastes by the present novel material can be done in a completely closed system, because burning up of those residues, land reclamation of their ashes and treatment of drainages such as prior arts can be canceled respectively.

Furthermore, the present novel material seems to be much effectively as an excelent medical drug because it shows that remarkable developments for not only dysfunction of the bioendocrine system and the bioimmunity system, but also prevention and herling of the life-style related diseses and the chromic diseases, appear on those biological tests respectively.

Various modifictions or substitutions of equivalent agricaltural and medical material may be made by those skilled in the art to the disclosed embodiment without departing from the scope and extent of the invention.

What is claimed is:

1. An adjuvant for accelating enzyme activity including many kinds of amino acid contained indispensable amino acids and many kinds of specific metalic cations characterized that organic substances and/or organic conpounds including a small quantity of inorganic subastances and showing a general formula; [CₐHₐO_{c}N_{d}PₑS_{f}] (but a-f indicating a positive number respectively), are contacted with catalaizers comprising microorganism group in nature, clay catalizer, enzymes nd water under atomospheric pressure, and proceeded oxidation-reductions through aerobic environments from anaerobic environments to yield the adjuvant.

2. An adjuvant for accelating enzyme activity in claim 1 wherein the oraganic substances and/or organic compounds are biomass materials.

3. An adjuvnt for accelating enzyme activity in claim 1 wherein the organic substances and/or organic compounds are bioresidures.
